(19) United States
(12) Patent Application Publication
(10) Pub. No.: US 2013/0143828 A1
(43) Pub. Date: Jun. 6, 2013

(54) GLYCOLIPID FOR TREATMENT OF ISCHEMIA REPERFUSION INJURY

(71) Applicant: Monica Molteni, Agrate Brianza (IT)

(72) Inventor: Monica Molteni, Agrate Brianza (IT)

(73) Assignee: BLUEGREEN BIOTECH S.R.L., Milano (IT)

(21) Appl. No.: 13/813,799

(22) PCT Filed: Aug. 2, 2011
(86) PCT No.: PCT/EP11/63281

§ 371 Date: Feb. 1, 2013

(30) Foreign Application Priority Data
Aug. 4, 2010 (IT) MI2010A001489

(51) Int. Cl. C07H 3/00

(52) U.S. Cl. CPC C07H 3/00
USPC 514/25; 536/18.2; 435/74

(57) ABSTRACT

The invention relates to a high molecular weight glycolipid characterized by the presence of rhamnose which has anti-inflammatory activity, particularly in inflammation triggered by ischemia and reperfusion. A further aspect of the invention is a process for preparation of said glycolipid from Cyanobacteria.

### FIELD OF THE INVENTION

The invention relates to a high molecular weight glycolipid characterized by the presence of rhamnose which has anti-inflammatory activity, particularly in inflammation triggered by the damage after ischemia and reperfusion injury.

A further aspect of the invention is a process for preparation of the glycolipid.

### STATE OF THE ART

Ischemia and reperfusion injury characterizes several pathologies including myocardial infarction, coronary heart disease, stroke and cerebral, cardiac, renal, intestinal, liver or lung ischemia and transplants. Damage occurs when ischemic tissue is temporarily deprived of blood supply, and the subsequent reperfusion, ie the subsequent restoration of blood flow in the tissue, causes an intense inflammatory response that may result in damage to organs not involved in the initial ischemic insult (Arumugam T V, et al., 2004, Shock 21:401-409). This represents a sterile inflammatory process, that is not associated with infection, which however determines the migration, adhesion and activation of immune cells at the affected site (S. Steffens et al. 2009, Thromb. Haemost., 102:240-247; Jang H. R. et al., 2009, Clin Immunol., 130:41-50; Lakhan S. E., et al. 2009, J. Transl. Med. 7: 97-107). Such activation involves the release of several pro-inflammatory mediators from immune cells, in addition it also involves the release from cells of damaged tissue of enzymes that alter the composition and structure of the extracellular matrix.

It is well known that extracellular release of ATP causes significant amplification of the inflammatory response, resulting in massive production of cytokines such as tumor necrosis factor alpha (TNF-alpha), interleukin (IL-) 6, IL-8 and IL-1 by cells of the innate immune system.

Extracellular release of ATP may cause irreversible damage not only of local tissue but also of the whole organ, and is at the basis of organ transplant failure resulting in rejection; it may also compromise the resolution of pathologies such as myocardial infarction or brain ischemia.

In addition, the intense inflammation triggered by ischemia/reperfusion (I/R), also as result of the extracellular release of ATP, may worsen patient's condition since it may cause the so-called “multiple organ dysfunction syndrome (MODS)” that may be fatal. Therefore, the ability to inhibit the pro-inflammatory loop caused by extracellular release of ATP is of fundamental importance in all pathologies characterized by a strong pro-inflammatory response induced by I/R in a tissue or an organ.

In particular, the necessity is felt to inhibit release of pro-inflammatory mediators, such as cytokines, when their production is stimulated by molecules produced locally in tissues subjected to ischemic stress.

The effects of lipopolysaccharides (LPS) derived from gram-negative bacteria have been studied for this purpose.

Pre-treatment with non-lethal doses of lipopolysaccharide prior to I/R induction has been shown to have potential protective effects against tissue damage in animal models of I/R in liver and heart (Meng X., et al. 1997, Am. J. Physiol. 273:H1894-902). LPS are molecules that stimulate pro-inflammatory cytokine production; when administered 24 hours before induction of I/R, they make the organism unable to withstand again, within a short time period, a strong pro-inflammatory response after I/R.

However, such approach in which LPS is administered 24 hours before induction of I/R is impractical in a clinical setting. In fact LPS treatment should be necessarily preventive, therefore cannot be applied in most pathologies involving damage from ischemia and reperfusion; moreover such treatment is debilitating for the patient because it mimics the immune activation that occurs in presence of a bacterial infection, thus causing fever and other symptoms. Finally it involves molecules that can cause a septic shock, and therefore can be lethal.

In addition to the effects of lipopolysaccharides from gram-negative bacteria, a cardioprotective effect of glycolipid RC-552 and monophosphoryl Lipid A (MPLA) has been shown (GT Elliot st al. 2000, J. Mol. Cell. Cardiology, 1327-39). These molecules have structural features similar to the active portion (Lipid A) of bacterial LPS, thus their mechanism of action also consists in preventive stimulation of the pro-inflammatory response.

Yet there is still a need to identify molecules that can act on extracellular release of ATP in order to avoid irreversible tissue damage and even damage to the whole organ.

Such molecules would be useful to prevent rejection of organ transplantation and resolve diseases such as myocardial infarction or brain ischemia.

The authors of this patent application previously described a glycolipid fraction from Cyanobacteria of the Order of Oscillatoriales that can antagonize the pro-inflammatory effects induced by exogenous bacterial molecules such as the LPS from E. coli (A. Macagno et al., 2006, J. Exp. Med. 203: 1481-92), Neisseria meningitidis (K. Jemmett, 2008, Infect. Immun. 76:3156-63) and P. gingivalis (PCT/E P2009/059565).

Therefore, said glycolipid fraction acts exclusively on cells of the innate immune system.

Therefore, the aim of the present invention is to identify a glycolipid that is capable of LPS antagonist activity and simultaneously is capable of inhibiting the synthesis of extracellular ATP by acting on the ATP synthase enzyme that is present on the plasma membrane of cells of the innate immune system, as well as of all other cell types.

Such synergistic activity results in a stronger overall anti-inflammatory activity that is also active on cells not belonging to the innate immune system.

### SUMMARY OF THE INVENTION

The present invention relates to a glycolipid of general formula (I):

(G)ₘ-(OCOR)ₙ (I)

wherein,
(G)ₘ: is the total saccharidic fraction;
(OCOR)ₙ: is the total lipidic fraction;
G is a saccharide;
R is a linear hydrocarbon chain comprising from 12 to 24 carbon atoms;
m is an integer from 50 to 150; and
n is an integer from 2 to 5;
   wherein G, comprises at least one rhamnose unit.

According to another aspect, the invention relates to the use of the glycolipid of the present invention as a medicament.

A further aspect of the invention relates to the use of the glycolipid according to the invention as anti-inflammatory agent.

Another aspect of the invention relates to the glycolipid according to the present invention for treatment of inflammation associated with ischemia and reperfusion.

A further aspect of the present invention relates to a process for preparation of the glycolipid comprising the steps of:
a. Providing a culture of at least one cyanobacterium;
b. Extracting the glycolipid from the culture;
c. Precipitating the glycolipid from step (b); and
d. Purifying the extracted and precipitated glycolipid from step (c).

A further aspect of the present invention relates to the glycolipid that can be obtained through the process of the present invention, having a molecular weight equal to or greater than 30 KDa.

### DESCRIPTION OF THE FIGURES

The invention will now be described in detail and with reference to the attached Figures, wherein:

FIG. 1 shows the results of two-dimensional electrophoretic analysis of the glycolipid of formula (I) according to the invention;

FIG. 2 shows a graphical representation of the inhibition by the glycolipid of formula (I) of IL-8 production induced by hypoxia in a human macrophage cell line;

FIG. 3A shows a graphical representation of the inhibition by the glycolipid of formula (I) of HSP70-induced IL-6 production in a human macrophage cell line;

FIG. 3B shows a graphical representation of the results of inhibition by the glycolipid of formula (I) of HSP70 induced TNF-alpha production in a human macrophage cell line;

FIG. 4 shows a graphical representation of the reduction by the glycolipid of formula (I) of infarction caused by ischemia and reperfusion in C57BL/6 mice;

### DETAILED DESCRIPTION OF THE INVENTION

Therefore the present invention relates to a glycolipid of general formula (I):

(G)ₘ-(OCOR)ₙ (I)

wherein,
(G)ₘ: is the total saccharidic fraction;
(OCOR)ₙ: is the total lipidic fraction;
G is a saccharide;
R is a linear hydrocarbon chain comprising from 12 to 24 carbon atoms;
m is an integer from 50 to 150; and
n is an integer from 2 to 5;
   wherein G, comprises at least one rhamnose unit.

In the present invention the definition:
“saccharidic fraction or saccharide” refers to a polysaccharide comprising from 50 to 150 saccharide units, wherein the glycosidic moiety may show the presence of aglycone substituents such as amino acids, phosphate and sulphate charged groups;
“glycolipid” refers to a compound (G)m-(OCOR)n comprising a saccharide fraction and a lipid fraction, wherein the lipidic fraction is covalently linked to the saccharidic fraction by an ester or amide bond, and
“lipidic or fatty acid fraction” are aliphatic monocarboxylic acids, wherein said monocarboxylic acid comprises a hydrocarbon chain with length ranging from 12 to 24 carbon atoms. Said fatty acids can be saturated or unsaturated if double bonds are absent or present in the carbon chain, respectively, moreover they can be hydroxylated if an OH group is present in their structure. Examples of such fatty acids are: lauric acid (dodecanoic), myristic acid (tetradecanoic), palmitic acid (hexadecanoic), margaric acid (heptadecanoic), stearic acid (octadecanoic), arachidic acid (eicosanoic), behenic acid (docosanoic), and lignoceric acid (tetracosanoic).

The amounts of G and R according to the invention are expressed in parts by weight with respect to 100 parts by weight, respectively of total (G)ₘ and (OCOR)ₙ fraction (100%).

According to a preferred aspect, said glycolipid also comprises galacturonic acid, mannose, galactose, glucose, glucosamine, xylose and combinations thereof, wherein these sugars can be simple or more preferably are complex and/or derivatives.

Preferably the glycolipid according to the present invention, wherein (G)ₘ comprises from 40 to 60% by weight of rhamnose (Rha), from 5 to 22% by weight of galacturonic acid (GalA), from 5 to 20% by weight of mannose (Man), from 5 to 20% by weight of galactose (Gal), from 1 to 15% by weight of glucose (Glc), from 1 to 15% by weight of glucosamine (GLcN), from 1 to 15% by weight of xylose (Xyl) with respect to the total saccharidic fraction (G)m (100%).

More preferably the glycolipid according to the present invention, wherein (G)m comprises from 48 to 54% by weight of rhamnose (Rha), from 10 to 18% by weight of galacturonic acid (GalA), from 8 to 14% by weight of mannose (Man), from 8 to 14% by weight of galactose (Gal), from 2 to 9% by weight of glucose (Glc), from 2 to 6% by weight of glucosamine (GLcN), from 2 to 6% by weight of xylose (Xyl) with respect to the total saccharidic fraction (G)ₘ (100%).

More preferably the glycolipid according to the present invention, wherein (G)ₘ comprises: rhamnose 50.8±0.6%, galacturonic acid 13.5%±2.4, xylose 3.7%±0.5, mannose 10.5%±1, galactose 11.1%±0.8, glucose 5.4%±2; glucosamine 4.3%±0.7. (data are expressed as mean±standard deviation).

The qualitative and quantitative analysis of saccharide residues can be done by GC-MS of acetylated methyl glycosides after hydrolysis of the glycolipid mixture with methanol and hydrochloric acid (HCl 1M/MeOH) for 20 h at 80° C., hexane extraction and acetylation with pyridine and acetic anhydride and/or by analyzing alditol acetates obtained after treatment with 2M trifluoroacetic acid for 1 h at 120° C., sodium borohydride treatment for 1 h and subsequent acetylation with pyridine and acetic anhydride.

Preferably R is a linear hydrocarbon chain comprising 18 carbon atoms [C₁₈].

In a preferred form, (OCOR)n comprises from 45 to 65% by weight with respect to the total lipidic fraction (OCOR)n (100%).

Preferably R is a linear hydrocarbon chain comprising 16 carbon atoms [C₁₆].

In a preferred form, (OCOR) n comprises from 20 to 45% by weight with respect to the total lipidic fraction (100%).

Preferably when R is a linear hydrocarbon chain containing 14, 15, 17, 19 or 20 carbon atoms [C_{Σ}], (OCOR)n comprises from 0 to 15% by weight with respect to the total lipidic fraction (OCOR) n (100%).

In a more preferred form, the composition of fatty acids comprises: C18:0 55%±7.5; C16:0+C16:3OH 34.9%±5.1; C15:30 H 7.6%±4.1; C20:0 1.7%±1.7 (data represent the mean±standard deviation of three independent samples).

The elemental analysis of the glycolipid showed the presence of 41% carbon, 48.5% oxygen, 6.5% hydrogen, 4% nitrogen.

Preferably the glycolipid according to the present invention, where R is a linear hydroxylated hydrocarbon chain and/or a linear saturated hydrocarbon chain.

The analysis of fatty acids can be performed by GC-MS after hydrolysis of the compound with methanol and hydrochloric acid (HCl 1M/MeOH) for 20 h at 80° C. and extraction in hexane phase as methyl esters, as better described in Example 2.

Preferably the glycolipid according to the present invention has a molecular weight equal to or lower than 30 kDa. More preferably, the glycolipid has a molecular weight from 30 to 45 KDa, more preferably the molecular weight is about 33 KDa, and comprises rhamnose.

Particularly preferred is the glycolipid according to the invention, wherein:
The molecular weight of said glycolipid is equal to or higher than 30 kDa;
(G)ₘ comprises from 48 to 54% by weight of rhamnose (Rha), from 10 to 18% by weight of galacturonic acid (GalA), from 8 to 14% by weight of mannose (Man), from 8 to 14% by weight of galactose (Gal), from 2 to 9% by weight of glucose (Glc), from 2 to 6% by weight of glucosamine (GLcN), from 2 to 6% by weight of xylose (Xyl) with respect to the total saccharidic fraction (G)ₘ (100%);
n is 2 and at least one R is [C₁₈] in an amount of 45 to 65% by weight with respect to the total lipidic fraction (OCOR)ₙ (100%) and/or is [C₁₆] in an amount of 20 to 45% by weight with respect to the total lipidic fraction (OCOR)ₙ (100%) and R is [C_{Σ}] in an amount of 0 to 15% by weight with respect to the total lipidic fraction (OCOR)ₙ (100%).

Particularly preferred is the glycolipid according to the invention, wherein:
The molecular weight of said glycolipid is equal to or higher than 30 kDa;
(G)ₘ comprises from 48 to 54% by weight of rhamnose (Rha), from 10 to 18% by weight of galacturonic acid (GalA), from 8 to 14% by weight of mannose (Man), from 8 to 14% by weight of galactose (Gal), from 2 to 9% by weight of glucose (Glc), from 2 to 6% by weight of glucosamine (GLcN), from 2 to 6% by weight of xylose (Xyl) with respect to the total saccharidic fraction (G)ₘ (100%);
n is 3, 4 or 5, and at least one R is [C₁₈] in an amount of 45 to 65% by weight with respect to the total lipidic fraction (OCOR)ₙ (100%) and/or is [C₁₆] in an amount of 20 to 45% by weight with respect to the total lipidic fraction (OCOR)ₙ (100%) and R is [C_{Σ}] in an amount of 0 to 15% by weight with respect to the total lipidic fraction (OCOR)ₙ (100%).

According to another aspect, the invention relates to the use of the glycolipid of the present invention as a medicament.

A further aspect of the invention relates to the use of the glycolipid according to the invention as anti-inflammatory agent.

In yet another aspect, the invention relates to the use of the glycolipid according to the present invention for the treatment of inflammation associated with ischemia and reperfusion.

The glycolipid has a high degree of purity, such purity being at least 90%, preferably at least 95%, and is capable of deactivating the pro-inflammatory response generated by molecules produced by the organism itself as a result of damage due to ischemia and reperfusion injury.

The present invention relates to a pharmaceutical composition having as active ingredient the glycolipid of formula I and a pharmacologically acceptable vehicle.

The total water solubility of the glycolipid makes possible its use in pharmaceutical compositions with aqueous excipients and diluents known to a technician in the field, especially with regard to liquid formulations.

The glycolipid, appropriately formulated into compositions, has anti-inflammatory activity when inflammation results from damage caused by ischemia and reperfusion injury, because said glycolipid inhibits the pro-inflammatory response induced by molecules that are formed during the process of ischemia and reperfusion, such as ATP.

The glycolipid of formula I is particularly useful in inflammation associated with ischemic events in tissues or organs, as in the case of myocardial infarction, coronary artery disease, cardiac arrest, atherosclerosis, stroke, brain, heart, kidney, intestine, liver or lung ischemia, transplantation and MODS.

Preferably, said inflammation is associated with ischemic events in tissues or organs, more preferably in the treatment of inflammation in myocardial infarction, coronary artery disease, cardiac arrest, atherosclerosis, stroke, cerebral ischemia, cardiac ischemia, renal ischemia, intestinal ischemia, liver ischemia, pulmonary ischemia, transplantation, multiple organ dysfunction syndrome.

Preferably the invention relates to a process for preparation of the glycolipid according to the present invention, comprising the steps of:
a. Providing a culture of at least one cyanobacterium;
b. Extracting the glycolipid from the culture;
c. Precipitating the glycolipid from step (b); and
d. Purifying the extracted and precipitated glycolipid from step (c).

More preferably, the invention relates to a process wherein said extraction (b) comprises a step of microwave treatment and a subsequent step of mixing with a denaturing solution, and said purification (d) comprises the elution from an anion exchange column and subsequent filtration through a filter with a cutoff of 30 KDa. Even more preferred is a process wherein such microwave treatment in step (b) is performed for about 1-2 minutes at about 750 W, said denaturing solution in step (b) comprises a reagent based on a polar protic organic solvent, a chaotropic agent and an aprotic organic solvent and said anion exchange column in step (d) involves a basic exchanger and said elution is performed with an ammonium bicarbonate gradient.

Preferably said culture of at least one cyanobacterium includes Oscillatoria sp. (No. 1459/45, CCAP Culture Collection of Algae and Protozoa, SAMS Research Services Ltd., Dunstaffnage Marine Laboratory, Dunbeg, Argyll, PA37 1QA, UK) According to a preferred embodiment of the procedure of the invention, said procedure involves the following steps:

After reaching the stationary growth phase, the cyanobacterial culture is sedimented for example by centrifugation (pelletting); the sediment (or bottom or pellet) can be freeze-dried before extraction.

Subsequently, the freeze-dried cyanobacteria are:
a) Rehydrated in an aqueous solution containing 25 mM Tris-HCl pH 7.5, deoxycholate (0.5%) and EDTA (5 mM), preferably in a volume comprised between 20 and 50 ml per gram of dry biomass;
b) Treated in microwave for 1-2 min at 750 W, in order to accelerate the process of glycolipid extraction;
c) Centrifuged preferably between 8000 and 10000×g to remove the pellet of cellular debris and recover the supernatant containing the glycolipid;
d) The so obtained extract (supernatant) is mixed, as described above, with a suitable volume of solution consisting of denaturing agents, eg. as described in Chomczynski P. and Mackey (BioTechniques, 1995, 19:942-5) preferably comprising a reagent based on a protic polar organic solvent, preferably phenol, and a chaotropic agent (such as guanidinium thiocyanate), for example.Tri reagent (Sigma cat. N T3934) or similar reagents, such as Trizol® (Invitrogen), and an aprotic organic solvent, such as for example chloroform or 1-bromo-3-chloropropane, in a ratio of about 1 volume of cyanobacterial supernatant, 1-2 volumes of extraction solution, preferably about 1, and chloroform (or 1-bromo-3-chloropropane), about 0.5-1 volume, in order to achieve greater purification of the glycolipid, favoring in particular the elimination of complexes consisting of photosynthetic pigments and associated proteins;
e) the extract is incubated at room temperature for at least 5 minutes, more preferably for a period of at least 10 minutes and less than 60′;
f) centrifugation, preferably at about 8000×g and collection of the supernatant (aqueous phase) containing polar glycolipids, which can be measured eg. by electrophoresis and silver-staining or staining with Pro-Q Emerald 300;
g) precipitation of the glycolipid present in the aqueous phase by addition of a salt, eg. sodium acetate (5-20 mM final) or magnesium chloride (150 mM) and an organic solvent, preferably acetone or ethanol, in amounts of about 2 volumes and incubation at 4° C. for at least 2 hours, preferably overnight;
h) centrifugation under the above conditions, recovery of the pellet and resuspension in water;
i) passage through a strong anion exchange column (basic exchanger: quaternary ammonium), for example Q75X (Sartorius) and elution with ammonium bicarbonate gradient, collection of the fraction eluted with 800 mM ammonium bicarbonate, containing the glycolipid;
j) repeated washing in water of the fraction thus obtained by use of a filter with a cut off of 30 KDa, and recovery of glycolipid molecules (which do not pass through the filter as they have a molecular weight greater than 30 KDa) at a high degree of purity, as assessed by measuring the presence of protein contamination, for example by the Bradford method, and nucleic acid contamination by spectrophotometric reading at 260 nm.

Without being bound to any theory, the procedure allows, due to the combined presence of denaturants and surfactants during microwave treatment, to mobilize structures of cyanobacterial cell wall and thus extract from such structures the hydrophilic components and macromolecular complexes, while removing the coarser cellular fragments (by centrifugation). Furthermore, the use of the microwave for cells rehydrated in aqueous solution, greatly favours and accelerates the release of the glycolipid molecules from the cells to the solution, increasing the final yield of product. The samples of glycolipid obtained with microwave treatment have also lower amounts of contaminant extracellular polysaccharides, containing glucose, than the samples obtained without this treatment. In fact, glucose content in the samples treated with microwaves (with respect to the total saccharidic component) was 5.4% compared to >10% in the samples without this treatment, thus having a key role in the final purity of the product. The glycolipid contained in the so obtained aqueous supernatant is purified by partitioning with organic solvents and chaotropic agents. This procedure is done to eliminate photosynthetic pigments and associated proteins partitioning in the phenolic phase. The procedure involves a subsequent passage on ion exchange column and elution with ammonium bicarbonate gradient. The eluate containing the active ingredient is placed on a filter with a molecular weight cut-off of 30 KDa, centrifuged and washed repeatedly in water. All the material below the cut-off limit is eliminated, while only the glycolipid remains above the filter.

A further aspect of the present invention relates to a glycolipid that can be obtained through the process of the present invention, having a molecular weight equal or higher than 30 kDa.

The glycolipid obtained by the process according to the present invention surprisingly has a high degree of purity, such purity being at least 90%, preferably at least 95%, and is capable of deactivating the pro-inflammatory response generated by molecules produced by the organism itself as a result of damage due to ischemia and reperfusion injury.

Surprisingly the microwave treatment greatly favours and accelerates the release of the glycolipid molecules from the cells to the solution, increasing the final yield of product. The glycolipid samples obtained by the process according to the present invention including the microwave treatment have also unexpected lower amounts of extracellular, glucose containing, polysaccharide contaminants, than the samples obtained without this treatment. In fact, glucose content in the samples treated with microwaves (with respect to the total saccharidic component) was 5.4% compared to >10% in the samples without this treatment, thus having a key role in the final purity of the product.

More preferably the glycolipid obtained by the process of the present invention includes rhamnose from 30 to 60% by weight with respect to the total saccharidic component (G)ₘ (100%).

### EXAMPLES

### Example 1

### Preparation of the Glycolipid of Formula I from Cyanobacteria

Cyanobacteria Oscillatoria sp., CCAP repository No. 1459/45 (made on Sep. 7, 2008 at the Centre of Culture Collection of Algae and Protozoa, Scotland, UK) were collected by centrifugation from the culture medium BG11 (cyanobacteria BG11 freshwater solution Cat. No. C3061, Sigma Aldrich) and freeze-dried. They were subsequently rehydrated with an aqueous solution containing Tris-HCl pH 7.5 (25 mM), deoxycholate (0.5%) and EDTA (5 mM) in a ratio of 30 ml per gram (g) of biomass and placed in microwave for 2 min at 750 W. The suspension was then centrifuged at 9000×g for 20 min and the supernatant containing the glycolipid was collected and incubated with 1 volume of chaotropic solution (TRI-Reagent, Sigma) and ½ volume of chloroform for 10 min at room temperature. After centrifugation at 8000×g for 10 min, the aqueous supernatant was collected and then incubated overnight at 4° C. in presence of sodium acetate (10 mM) and 2 volumes of acetone. After centrifugation at 8000×g for 15 min, the pellet containing the glycolipid was dried and then resuspended in water.

Purification of the active ingredient was then carried out by anion-exchange chromatography (Sartorius) and gradient elution with ammonium bicarbonate. The glycolipid was eluted with 800 mM ammonium bicarbonate and then placed on a filter with 30 KDa molecular weight cut-off and washed with MilliQ water, by subsequent centrifugations. The product was recovered from the upper part of the filter by addition of water and was then lyophilized. The sample was then resuspended in water or saline for subsequent chemical and biological tests. Cyanobacterium can be grown in a culture medium, consisting for example of BG-11 (Sigma-Aldrich, Cat No. C3061), at a temperature between 25° C. and 27° C. in presence of a cool white light at an intensity of 100 μmol. photons m-2.sec-1, under continuous light during 24 h.

### Example 2

### Chemical Analysis of the Glycolipid by Gas Chromatography and Mass Spectrometry Technique (GC-MS or Gas Chromatography—Mass Spectrometry)

The glycolipid extracted and purified as described in Example 1 is characterized by low protein contamination (lower than 1%), detectable by the Bradford method, and less than 3% nucleic acid contamination, as determined by spectrophotometric analysis.

Qualitative and quantitative analysis of saccharide residues was performed by GC-MS of acetylated methyl glycosides and alditol acetates; likewise fatty acids were detected as methyl esters by GC-MS through analysis of the retention times of chromatographic peaks and fragmentation of the mass spectra.

### Methods:

In detail, an aliquot of the sample (1 mg) was treated with methanol and hydrochloric acid (HCl 1M/MeOH) for 20 h at 80° C. and was subsequently dried and extracted with hexane; the hexane phase contains fatty acids as methyl esters, while the methanol phase contains O-methyl glycosides.

Acetylated methyl-glycosides were prepared as follows: the methanol phase was dried under air flow and acetylated with 50 μl of pyridine and 50 μl of acetic anhydride at 100° C. for 30 min. The mixture was dried, dissolved in CHCl₃, extracted several times with water in order to purify the sample, and then recovered and dried.

For alditol acetates an identical sample aliquot was treated with 2M trifluoroacetic acid at 120° C. for one hour. After drying the acid, the sample was taken up in water and treated for an hour with a spatula tip of sodium borohydride. Excess hydride was destroyed with acetic acid and the solution was dried several times with methanol and acetic acid. Finally, acetylation is carried out as for acetylated methyl glycosides. Both acetylated methyl glycosides and alditol acetates were analyzed by GC-MS.

The result of the analysis of acetylated methyl glycosides and alditol acetates can be viewed in chromatograms where each derivatized monosaccharide shows its own retention time; a mass spectrum typical of each monosaccharide is associated with each peak. The area under the peaks is proportional to the amount of monosaccharide present in the mixture. The analysis of alditol acetates makes possible to confirm and complete the panel of detected monosaccharides, with the additional advantage of providing a unique signal for each monosaccharide.

From the analysis of three independent samples, the following saccharides were detected (data are expressed as mean±standard deviation):

rhamnose 50.8±0.6%, galacturonic acid 13.5%±2.4, xylose 3.7%±0.5, mannose 10.5%±1, galactose 11.1%±0.8, glucose 5.4%±2; glucosamine 4.3%±0.7.

Lipids obtained after treatment of the compound with hydrochloric acid and methanol and hexane extraction, in order to separate them from methyl glycosides, were analyzed by GC-MS, which gave the following fatty acid composition (data represent the mean±standard deviation of three independent samples):

C18:0 55%±7.5; C16:0+C16:30 H 34.9%±5.1; C15:3OH 7.6%±4.1; C20:0 1.7%±1.7

The elemental analysis performed on the glycolipid revealed the presence of carbon 41%, oxygen 48.5%, hydrogen 6.5%, nitrogen 4%.

### Molecular Weight of the Glycolipid:

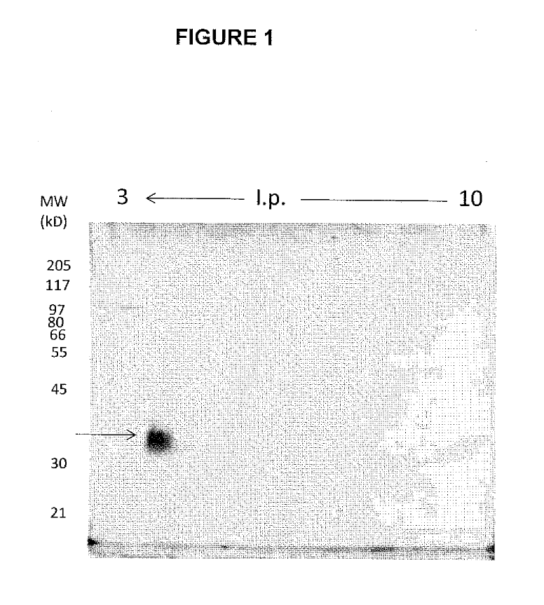

pH gradient two-dimensional electrophoresis and molecular weight separation in 10% Bis-Tris Novex NuPAGE gel (Invitrogen), followed by staining with Pro-Q Emerald 300 (Invitrogen, Molecular Probes cat. P20495) specific for polar glycolipid structures such as LPS, showed that the sample (5 μg loading) migrates as single spot with molecular weight slightly higher than 30 KD and an isoelectric point at acidic pH (FIG. 1).

The glycolipid is soluble in water, the solution is clear, colorless, odorless, tasteless. The compound is stable after boiling for 5 min or after one freeze-thaw cycle.

### Example 3

### Inhibition by Glycolipid of Formula I of IL-8 Production Induced by Hypoxic Treatment of Human Macrophages

During ischemia, tissues undergo hypoxic stress caused by low oxygen concentration. Macrophages accumulate in ischemic areas proportionally to the degree of cell necrosis in the tissue, and their activation is associated with release of a series of pro-inflammatory mediators, including IL-8, IL-6, TNF-alpha, which serve to recruit polymorphonuclear leukocytes and monocytes at the site of damage during the following reperfusion phase, thereby amplifying the inflammatory response. It is possible to mimic in vitro the effects of low oxygen concentration on macrophage activation, by incubating directly the cells in hypoxic environment. Hypoxic treatment of macrophages for 24 hours induces IL-8 release into the culture medium, while the levels of IL-8 are significantly inhibited in presence of the glycolipid of formula I.

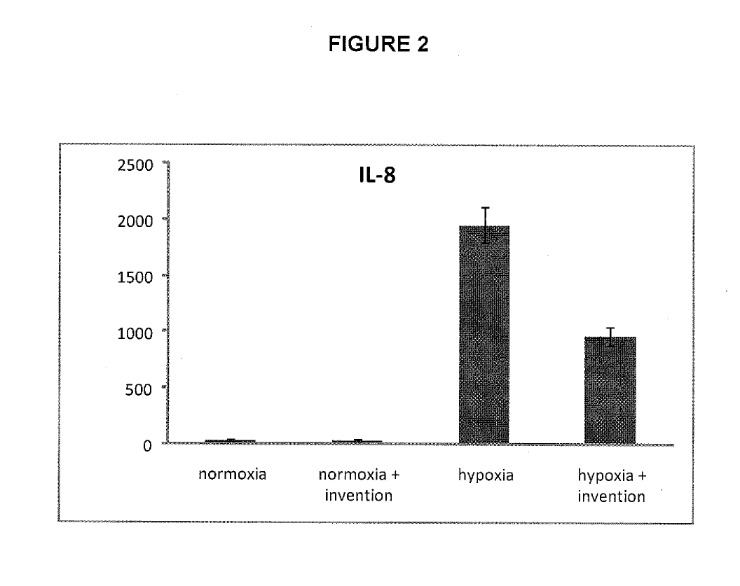

The glycolipid of formula I, prepared as described in Example 1, was used in vitro to study the effects on IL-8 production by macrophages incubated in hypoxic conditions for 24 hours. The THP1 human monocytic cell line (T. Oda et al., 2006, Am J. Physiol. Cell Physiol. 291:104-113) was differentiated in vitro (1×10⁵ cells/well in 96-well culture plate) by treatment for 48 hours with 5×10⁻⁸M phorbol myristate acetate (PMA) followed by incubation for 24 hours in absence of other stimuli before hypoxic treatment. Cells were then incubated with or without glycolipid (20 ug/ml) and placed in normoxia at 37° C. in 5% CO2 (control) or in hypoxic conditions at 37° C. using anaerobic AnaeroGen bags (Oxoid, UK) that allow incubation of cultures at an oxygen concentration lower than 1%. Supernatants were collected after 24 hours for quantitative assessment of IL-8 concentration. IL-8 was assessed by ELISA (Diaclone, human IL-8 Cat. No. 851530001) and results showed that IL-8 production was close to zero under normoxic conditions, both in presence and absence of cyanobacterial glycolipid. Hypoxic treatment leads to increased IL-8 levels, which are however significantly inhibited if the glycolipid of formula I is present in culture (FIG. 2. Data represent the mean±SD of 3 experiments).

It is therefore evident that treatment with the glycolipid is of critical importance in reducing the overall pro-inflammatory response associated with ischemia.

### Example 4

### Inhibition by the Glycolipid of IL-6 and TNF-Alpha Pro-Inflammatory Cytokine Production in Human Macrophages Induced by Heat Shock Protein (HSP) 70

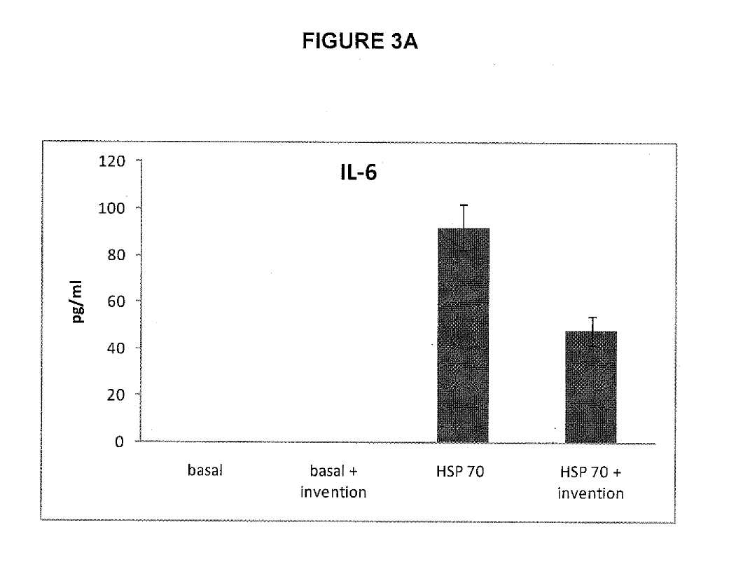

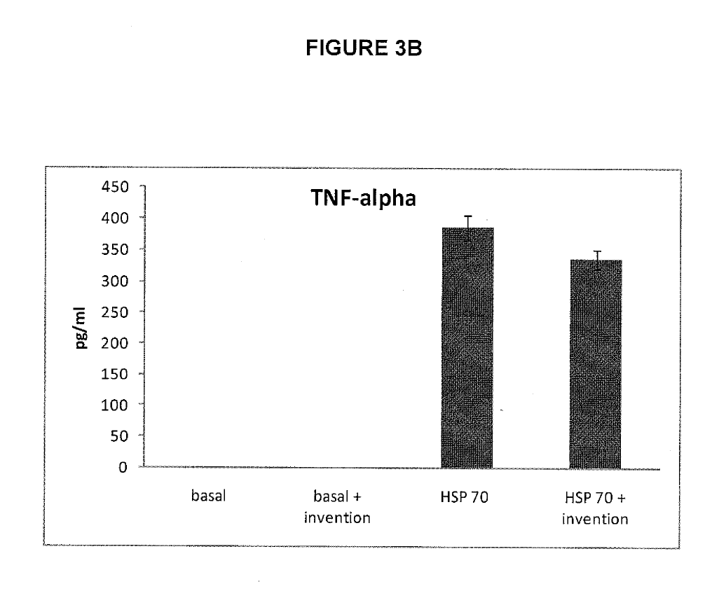

The THP1 human monocytic cell line differentiated as described above was stimulated with 10 ug/ml recombinant HSP70 (Stressgen, low endotoxin recombinant protein level cat. No. ESP-555) in presence or absence of the glycolipid of formula I at a concentration of 20 μg/ml. Cultures were incubated for 18 hours at 37° C. in 5% CO₂, and then supernatants were collected to assess IL-6 and TNF-alpha concentrations (FIG. 3A, 3B). Cytokines were analyzed by the ELISA test (Diaclone, human IL-6 Cat. No. 851,520,005, human TNF-alpha cat. No. 851570005). As shown in FIG. 3A, results (mean±SD) showed that the glycolipid of formula I inhibits IL-6 production and, as shown in FIG. 3B, TNF-alpha production.

### Example 5

### Assessment of the In Vivo Tolerable Dose of the Glycolipid of Formula I

Before proceeding with experiments on in vivo cardiac I/R injury, preliminary experiments were performed to evaluate the non-toxic range of the glycolipid of formula I and to rule out any possible hypotensive effect of the injected solution. These experiments were performed in rats by injecting the compound intravenously up to a dose of 75 mg/kg in saline. Results showed that the dose of 75 mg/kg is absolutely non-toxic, well tolerated and has no hypotensive effect on the circulatory system.

### Example 6

### Assessment of the Anti-Inflammatory Activity of the Glycolipid of Formula I In Vivo in Mice with Acute Myocardial Infarction Caused by Coronary Artery Ligation, Followed by Reperfusion (I/R)

Induction of acute myocardial infarction was performed in C57BL/6 mice, as described by M. Salio et al., Circulation, 117:1055-1064, 2008. Animals were divided into 2 groups: a control group, a group treated with the glycolipid of formula I dissolved in PBS administered at a dose of 10 mg/kg ip 30 min prior to I/R followed by 5 mg/kg i.v. at reperfusion. The control group was treated in the same way with PBS only.

Animals were anesthetized (Sigma Aldrich, Avertin, 250 mg/kg, ip), then intubated with a 22G cannula and ventilated, keeping the body temperature constant at 37±2° C. Thoracotomy was then performed at the level of the fourth intercostal space to expose the heart, the left anterior descending coronary artery was temporarily closed with a suture around the artery to produce the occlusion. The tourniquet was removed after 45 min of ischemia. Pneumothorax was then reduced and animals were allowed to recover. Postoperative analgesia was performed with buprenorphine (0.1 mg/kg sc q 12 hours for 1 day), and ampicillin (150 mg/kg) was administered after surgery. Mice were sacrificed at 24 hours after induction of ischemia.

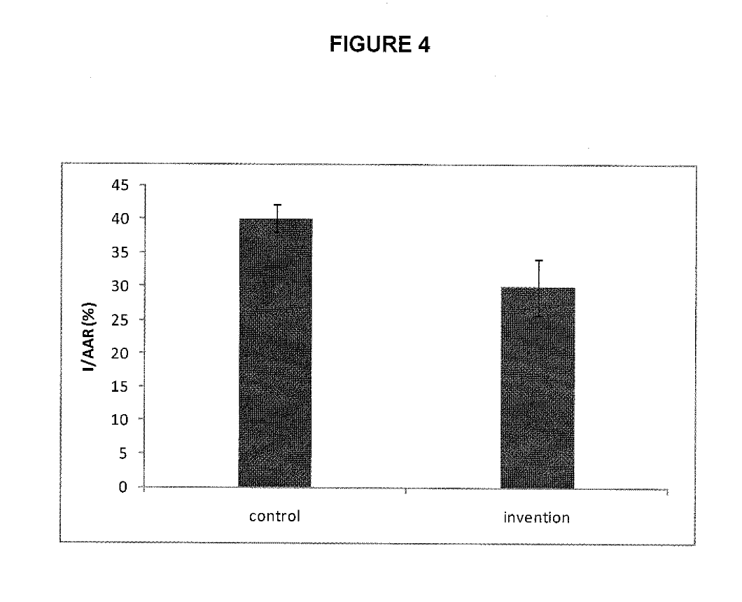

To measure infarct size, the Evans Blue dye was injected into the inferior vena cava, and left and right cardiac ventricles were separated. The left ventricle was then transversely sectioned in 1 mm thick slices which were incubated (for 20 min at 37° C.) in solution (PBS) containing tetrazolium chloride (TTC, Sigma Aldrich) and were then transferred to 4% formalin and incubated overnight prior to image analysis (Analytical Imaging Station, version 3.0, Imaging Reseach, St. Catherine's, ON Canada). Non-infarcted areas which were previously subjected to ischemia (areas at risk, AAR) appear red, while the infarcted tissue (I) appears white. The extension of the areas at risk was comparable in control animals and animals treated with the glycolipid of formula I. Results showed that treatment with the glycolipid of formula I significantly reduces the infarct size within the area at risk (I/AAR) compared to controls (FIG. 4), with 25% average reduction (the experiment was done with 10 control animals and 10 animals treated with the glycolipid of formula I).

1. A glycolipid of general formula (I):
(G)ₘ-(OCOR)ₙ (I)
wherein,
(G)ₘ: is the total saccharidic fraction;
(OCOR)ₙ: is the total lipidic fraction;
G is a saccharide;
R is a linear hydrocarbon chain comprising from 12 to 24 carbon atoms;
m is an integer from 50 to 150; and
n is an integer from 2 to 5;
wherein G, comprises at least one rhamnose unit.

2. The glycolipid according to claim 1, wherein G comprises at least one saccharide unit selected from the group consisting of galacturonic acid, mannose, galactose, glucose, glucosamine, xylose and combinations thereof.

3. The glycolipid according to claim 1, wherein (G). comprises from 48 to 54% by weight of rhamnose (Rha), from 10 to 18% by weight of galacturonic acid (GalA), from 8 to 14% by weight of mannose (Man), from 8 to 14% by weight of galactose (Gal), from 2 to 9% by weight of glucose (Glc), from 2 to 6% by weight of glucosamine (GLcN), from 2 to 6% by weight of xylose (Xyl) with respect to the total saccharidic fraction (G)ₘ (100%).

4. The glycolipid according to claim 1, wherein R is a linear hydrocarbon chain comprising 18 carbon atoms [C₁₈] and wherein (OCO[C₁₈])ₙ is 45 to 65% by weight with respect to the total lipidic fraction (OCOR)ₙ (100%).

5. (canceled)

6. The glycolipid according to claim 4, wherein R is a linear hydrocarbon chain comprising 16 carbon atoms [C₁₆] and wherein (OCO[C₁₆])ₙ is 20 to 45% by weight with respect to the total lipidic fraction (OCOR)ₙ (100%).

7. (canceled)

8. The glycolipid according to claim 1, wherein when R is a linear hydrocarbon chain comprising 14, 15, 17, 19 or 20 carbon atoms [C_{Σ}], (OCO[C_{Σ}])ₙ is 0 to 15% by weight with respect to the total lipidic fraction (OCOR)ₙ (100%).

9. The glycolipid according to claim 1 having a molecular weight equal to or higher than 30 kDa.

10. The glycolipid according to claim 1, wherein:
The molecular weight of said glycolipid is equal to or higher than 30 kDa;
(G)ₘ comprises from 48 to 54% by weight of rhamnose (Rha), from 10 to 18% by weight of galacturonic acid (GalA), from 8 to 14% by weight of mannose (Man), from 8 to 14% by weight of galactose (Gal), from 2 to 9% by weight of glucose (Glc), from 2 to 6% by weight of glucosamine (GLcN), from 2 to 6% by weight of xylose (Xyl) with respect to the total saccharidic fraction (G)ₘ (100%);
n is 2 and at least one R is [C₁₈] in an amount of 45 to 65% by weight with respect to the total lipidic fraction (OCOR)ₙ (100%) and/or is [C₁₆] in an amount of 20 to 45% by weight with respect to the total lipidic fraction (OCOR)ₙ (100%) and R is [C_{Σ}] in an amount of 0 to 15% by weight with respect to the total lipidic fraction (OCOR)ₙ (100%).

11. The glycolipid according to claim 1, wherein:
The molecular weight of said glycolipid is equal to or higher than 30 kDa;
(G)ₘ comprises from 48 to 54% by weight of rhamnose (Rha), from 10 to 18% by weight of galacturonic acid (GalA), from 8 to 14% by weight of mannose (Man), from 8 to 14% by weight of galactose (Gal), from 2 to 9% by weight of glucose (Glc), from 2 to 6% by weight of glucosamine (GLcN), from 2 to 6% by weight of xylose (Xyl) with respect to the total saccharidic fraction (G)ₘ (100%);
n is 3 and at least one R is [C₁₈] in an amount of 45 to 65% by weight with respect to the total lipidic fraction (OCOR)ₙ (100%) and/or is [C₁₆] in an amount of 20 to 45% by weight with respect to the total lipidic fraction (OCOR)ₙ (100%) and R is [C_{Σ}] in an amount of 0 to 15% by weight with respect to the total lipidic fraction (OCOR)ₙ (100%).

12. The glycolipid according to claim 1, wherein:
The molecular weight of said glycolipid is equal to or higher than 30 kDa;
(G)ₘ comprises from 48 to 54% by weight of rhamnose (Rha), from 10 to 18% by weight of galacturonic acid (GalA), from 8 to 14% by weight of mannose (Man), from 8 to 14% by weight of galactose (Gal), from 2 to 9% by weight of glucose (Glc), from 2 to 6% by weight of glucosamine (GLcN), from 2 to 6% by weight of xylose (Xyl) with respect to the total saccharidic fraction (G)ₘ (100%);
n is 3, 4 or 5, and at least one R is [C₁₈] in an amount of 45 to 65% by weight with respect to the total lipidic fraction (OCOR)ₙ (100%) and/or is [C₁₆] in an amount of 20 to 45% by weight with respect to the total lipidic fraction (OCOR)ₙ (100%) and R [C_{Σ}] in an amount of 0 to 15% by weight with respect to the total lipidic fraction (OCOR)ₙ (100%).

13. A pharmaceutical composition comprising the glycolipid according to claim 1 and at least pharmaceutically acceptable excipient.

14. A method for treating an inflammatory condition comprising administering to a subject in need thereof a glycolipid according to claim 1.

15. The method according to claim 12 wherein said inflammatory condition is associated to ischemia and reperfusion.

16. The method according to claim 12, wherein said inflammatory condition is associated to tissue ischemia and organ ischemia.

17. The method according to claim 12, wherein said inflammatory condition is associated to myocardial infarction, coronaropathy, heart failure, atherosclerosis, cerebral ischemia, cardiac ischemia, renal ischemia, intestinal ischemia, hepatic ischemia, pulmonary ischemia, transplantation and multiple organ dysfunction syndrome.

18. A process for the preparation of the glycolipid according to anyone of claim 1, comprising the steps of:
a. Providing a culture of at least one cyanobacterium;
b. Extracting the glycolipid from the culture;
c. precipitating the glycolipid from step (b); and
d. purifying the extracted and precipitated glycolipid from step (c).

19. Process according to claim 18, wherein said extraction (b) comprises a step of treatment with microwaves and a successive step of mixing with a denaturing solution, and said purification (d) comprises elution on an anion exchange column and a successive filtration with a filter having a cut off at 30 KDa.

20. Process according to claim 19, wherein said treatment with microwaves of step (b) is carried out for about 1-2 min at about 750 W, said denaturing solution of step (b) comprises an organic polar protic solvent based reagent, a caotropic agent, and an aprotic organic solvent and said anion exchange column of step (d) has a basic exchanger and said elution is carried out with and ammonium bicarbonate gradient.

21. Process according to anyone of claim 18 to 20, wherein said culture of at least one cyanobacterium comprises Oscillatoria sp. (N° 1459/45, CCAP).

22. A glycolipid obtainable by the process according to anyone of claims from 18 a 21 and having a molecular weight equal to or higher than 30 kDaᵥ, comprising from 30 to 60% by weight of rhamnose with respect to the total saccharidic fraction (G)ₘ (100%).

23. (canceled)
